(19) United States
(12) Patent Application Publication
(10) Pub. No.: US 2005/0159794 A1
(43) Pub. Date: Jul. 21, 2005

(54) Multiple port photodynamic therapy irradiation system

(75) Inventor: Wolfgang Neuberger, Labuan (MY)

Correspondence Address: BOLESH J. SKUTNIK PhD. JD, 515 Shaker Road, East Longmeadow, MA, 01028, US

(73) Assignee: CeramOptec Industries, Inc.

(21) Appl. No.: 10/757,910

(22) Filed: Jan. 15, 2004

(51) Int. Cl. A61N 5/00; A61N 1/00

(52) U.S. Cl. 607/88; 607/89

(57) ABSTRACT

A diode-based radiation source is disclosed, having multiple output ports for delivery of radiation to multiple delivery devices such as optical fibers. The radiation source, useful especially for photodynamic therapy and interstitial tumor therapy, contains a number of diode units, wherein a number of individual emitters' output powers are combined to each output port. A computer or other control unit enables the desired output power to be adjusted separately for each port and enables each delivery system to be calibrated individually. In this way, multiple delivery devices can be inserted into a treatment area from a single source and be individually adjusted to provide a desired distribution of radiation power in the treatment area.

### BACKGROUND OF THE INVENTION

1. Field of the Invention

The present invention relates to the field of radiation sources for medical application, especially photodynamic therapy and interstitial radiation therapy. In particular, the present invention relates to multiple output laser sources.

2. Information Disclosure Statement

Photodynamic therapy (“PDT”) has been proven effective for a variety of treatments, most notably cancer and skin diseases such as psoriasis. The general procedure is as follows: a photosensitive drug (photosensitizer), which is activatable by radiation of a certain wavelength, is administered to a diseased or otherwise affected area of the body. Sufficient time is allowed to pass for the photosensitizer to accumulate in hyperproliferating tissue and be substantially flushed out of other areas of the body. After this period, radiation of a certain wavelength is applied to the treatment area to activate the photosensitizer. This activation causes the photosensitizer to become cytotoxic. The strength of PDT lies in its selectivity, particularly in the fact that it preferentially accumulates in hyperproliferating tissue such as cancerous tissue and has a limited-depth range of effect due to the limited penetration depth of the activation wavelength used. Preferential accumulation of the photosensitizer is rarely sufficient by itself to generate the required selectivity; the combination of both preferential accumulation of photosensitizer and limited radiation penetration depth allows the cytotoxic effect generated by activating the photosensitizer to be substantially limited to diseased tissue.

For this reason, it is advantageous in photodynamic therapy and other radiation treatments to controllably apply and restrict radiation to certain areas in order to minimize risk to damage of healthy tissue.

A variety of methods of irradiating tissue are known that attempt to restrict radiation to certain body areas, as well as various methods for introducing radiation to internal body areas that cannot be reached safely by external irradiation. Optical fibers are extremely important in this regard, due to their ability to guide radiation. Optical fibers are used to guide radiation through body cavities, blood vessels, and directly into tissue.

One advantageous method, especially for tumors and particularly useful in interstitial PDT applications, is to simultaneously or sequentially irradiate a tumor or other tissue mass with numerous optical fibers inserted in various positions within the mass to ensure a specific distribution of radiation within the tissue. Application of radiation can be more tightly controlled in such a method, as individual fibers can deliver relatively low powers, reducing the amount of radiation that could affect surrounding healthy tissue.

U.S. Pat. No. 4,957,481 describes methods for reduction of symptoms associated with tumors, such as pain caused by pressure on a nerve from a tumor mass, and for reduction of tumor size. The methods entail administration of a photosensitizer directly into a tumor mass and irradiation from at least one sheathed light source directly injected into the tumor to activate the photosensitizer and disrupt the tumor. Administration of the photosensitizer can be accomplished by a single intratumoral injection near the center of the tumor, or multiple direct administrations can be applied to improve diffusion of the photosensitizer within the tumor. Irradiation can be accomplished by a single light source (such as an optical fiber coupled to a laser) inserted into the tumor, multiple light sources inserted into the tumor, or a single light source inserted numerous times and at numerous locations within the tumor. This patent does not disclose a radiation source unit having numerous ports capable of providing radiation simultaneously to multiple delivery devices.

Generally, laser units used with multiple laser delivery systems consist of a single output port and a beam splitting device that distributes the total output power to a certain number of delivery devices. Other devices disclosed for interstitial irradiation are disclosed that insert actual radiation sources directly into tissue.

U.S. patent application No. 2002/0010500 and U.S. Pat. No. 6,416,531 disclose a method for administering light at multiple treatment sites within a tumor. A plurality of fibers or light-emitting probes are deployed within the tumor in a spaced apart array. Light is administered to activate photosensitizers absorbed within the tumor and destroy the cancerous tissue. In one embodiment, a light source is fitted with a splitter to divide the emitted beam into distinct beams which are guided to different areas within the tumor by a plurality of optical fibers. Other embodiments describe implantable probes having light sources such as LEDs that emit light from within each probe.

U.S. Pat. No. 6,099,554 discloses a laser light delivery method and systems that utilizes multiple laser beams to simultaneously irradiate a tumor mass from a variety of directions. An array of multiple light sources is used, purportedly to minimize exposure to healthy tissue and homogenize applied radiation. In one embodiment, a plurality of optical fibers are positioned to surround a tumor and simultaneously irradiate it from all sides. This invention requires the use of numerous radiation source units. It does not disclose a single device capable of delivering radiation to multiple fibers or other delivery devices.

U.S. Pat. No. 6,048,359 describes a medical apparatus including multiple implantable probes for providing spatially oriented radiation delivery into tissue for diagnosis and photodynamic therapy. Multiple probes, each inserted into tissue, contain light sources such as LEDs for providing diffused light as a uniform dosage within the tissue.

Methods and devices are known for controlling numerous laser devices, although there has not been disclosed an apparatus suitable for separately controlling radiation delivery devices in a single unit that is suitable for interstitial radiation treatments.

U.S. Pat. No. 6,445,670 describes a laser power control device for controlling the output of laser light from multiple sources, for use in optical recording and reproduction. A system of resistors, a photodetector and an output stabilizer acts to individually set and maintain the output power of each diode light source, allowing separate wavelengths to be applied to an optical recording system in a single unit. This device is described for optical recording systems. It does not disclose a plurality of diode laser units each having different power ranges and applicable to medical treatments. It further does not disclose a laser device having multiple output ports allowing individual light sources to be coupled to separate waveguides.

A laser diode power supply, stabilizer and controller for use in communication systems and optical memory devices is disclosed in U.S. Pat. No. 5,604,757 that controls and regulates the parameters of multiple laser diodes. Each laser diode is separately regulated to control parameters such as current, power and temperature regulation. This invention is primarily concerned with reducing noise associated with powering multiple diodes with a single power supply, and it does not disclose a medical diode laser treatment apparatus having means to separately connect different diode lasers or diode laser arrays to separate waveguides. It further does not provide means to transmit radiation from multiple sources having different power ranges from a single apparatus. Also, this invention and U.S. Pat. No. 6,445,670 (described above) describe means to only adjust individual single diodes. This is not preferable, in that, although diode powers can be adjusted within a given range, there is no ability to adjust the power from each light source beyond the limited range inherent in the diode.

There are numerous disadvantages inherent in prior art systems and devices for interstitial radiation therapy. Devices that utilize splitters to delivery radiation to numerous delivery devices lack the ability to individually control the power output from each delivery device. Systems that utilize numerous sources are needlessly cumbersome and can prove expensive. Devices or systems that utilize light sources in probes directly inserted into tissue lack flexibility, in that sources cannot be replaced (whether due to malfunction or the desire to incorporate higher power sources) without the need for removal and re-sterilization of the probes.

There has not been disclosed an integrated laser radiation source that allows a user to apply radiation with potentially different power ranges to multiple positions simultaneously while being able to individually control and calibrate each delivery point with the single source. The present invention addresses this need and overcomes other disadvantages of the prior art.

### OBJECTIVES AND BRIEF SUMMARY OF THE INVENTION

It is an object of the present invention to provide a radiation source allowing multiple radiation delivery systems to be applied simultaneously.

It is another object of the present invention to provide a radiation source useful for photodynamic therapy or interstitial radiation therapy capable of supplying radiation to multiple interstitial delivery systems.

It is yet another object of the present invention to provide a radiation source with multiple output ports that can be individually adjusted to a preselected power level.

It is still another object of the present invention to provide a radiation source having multiple outputs, wherein each output is capable of potentially providing distinct power ranges.

It is another object of the present invention to enable the individual calibration of each output port and each device linked to said port.

Briefly stated, the present invention describes a diode-based radiation source having multiple output ports for delivery of radiation to multiple delivery devices such as optical fibers. The radiation source, useful especially for photodynamic therapy and interstitial tumor therapy, contains a number of diode units, wherein a number of individual emitters' output powers are combined to each output port. A computer or other control unit enables the desired output power to be adjusted separately for each port and enables each delivery system to be calibrated individually. In this way, multiple delivery devices can be inserted into a treatment area from a single source and be individually adjusted to provide a desired distribution of radiation power in the treatment area.

The above, and other objects, features and advantages of the present invention will become apparent from the following description read in conjunction with the accompanying drawings, in which like reference numbers in different drawings designate the same elements.

### BRIEF DESCRIPTION OF FIGURES

FIG. 1—An illustration of the exterior of an embodiment of the present invention.

FIG. 2—An illustration of the interior of an embodiment of the present invention.

FIG. 2a—A close-up view of the interior of an embodiment of the present invention.

### DETAILED DESCRIPTION OF PREFERRED EMBODIMENTS

The present invention provides a diode based system, for use in treatments such as photodynamic therapy, wherein multiple delivery devices can be attached to a multitude of output ports and wherein the individual power of each output port (and thus each delivery device) can be chosen within a certain predetermined range. For example, one or more ports may be chosen to emit high maximum power, such as 12 W, whereas other ports may preferably be limited to lower power ranges having maximums of 1 W, 5 W or other levels.

Photodynamic therapy (“PDT”) is an effective treatment for hyperproliferative diseases such as psoriasis and cancer. One obstacle to certain cancer treatments occurs when tumors are located deep within the body. In some cases, optical fibers or optical fiber bundles can effectively reach cancerous tissue located within body lumens, such as the intestines, and provide radiation that can penetrate into diseased tissue and activate photosensitizers to provide a photo-cytotoxic effect.

In other cases, tumors within the body are not so easily reached, though in some cases fibers or fiber bundles can be inserted directly into the tumor mass. Often, due to the size of the tumor or the absorptive properties of the tissue relative to the treatment wavelength, radiation from a single source is not sufficient to provide energy of sufficient power to all areas of the tumor. In such cases, treatments have been performed consisting of the insertion of multiple interstitial radiation delivery devices, spatially positioned so as to provide radiation of sufficient power throughout the diseased tissue. This treatment is known as interstitial radiation therapy or interstitial tumor therapy, which encompasses both irradiation therapy without the use of photosensitizers and photodynamic therapy.

It can be very cumbersome to provide a separate radiation source for each delivery system in interstitial PDT or other interstitial laser treatments, and for this reason, systems have been developed for this type of treatment wherein radiation can be delivered to multiple delivery systems simultaneously. Traditionally, the task of irradiating a tumor with multiple interstitial delivery systems has been handled with a laser unit having a simple output port and a beam splitter that distributes the total output power to a predetermined number of ports. The disadvantage with this setup is that significant amounts of laser power are lost in the beam splitter and the powers of the devices can usually be regulated only simultaneously and not independently from each other. Other proposed solutions include systems having numerous probes wherein the light source is disposed directly within the probe and emits radiation directly into tissue.

There are numerous additional advantages of having multiple radiation sources within the treatment unit, rather than having multiple sources disposed within the body. If an individual source such as a diode or diode array fails or is defective, it can be quickly and easily replaced without the need to remove any devices from the tissue. The present invention also makes it easier to increase the maximum power by replacing the type of diode emitter or by adding additional diode emitters to a diode unit, potentially without the need to remove and replace devices within tissue and without the need for additional sterilization.

The present invention provides a single radiation source unit especially useful for interstitial tumor PDT or interstitial tumor therapy that has the ability to provide radiation to multiple delivery systems simultaneously and without the loss of power typical of devices that split radiation from a source. Additionally, the present invention is advantageous in that the power to each individual delivery device can be set or controlled independent of one another. Because the user can set the power range for each output port, the user is essentially unlimited in the radiation distribution patterns that are possible.

The ability to independently control the radiation power has significant effects in producing a treatment that can effectively irradiate diseased tissue while leaving healthy tissue as unaffected as possible. Radiation unintentionally applied to healthy tissue can cause significant damage to healthy tissue, whether through thermal damage or interaction with any remaining photosensitizer that has not completely left the tissue. For these reasons, it is important to reduce the amount of radiation that affects healthy tissue.

For example, when a tumor is treated with PDT, it is important to minimize the amount of healthy tissue around the periphery of a tumor that is irradiated, for the reasons described above. There may be a need to irradiate some of the peripheral healthy tissue as a safety mechanism to ensure that no cancer cells survive, but this of course should be controlled. When a plurality of fibers are placed within a large tumor, relatively high powers should be applied to central areas of the tumor, to minimize the number of insertions required in order to minimize trauma or pain to the patient. Toward the periphery of the tumor, however, it would be important to utilize lower power fibers that would exhibit a smaller range of effect, in order to avoid irradiating too much healthy tissue. In the prior art, such a treatment setup would require numerous lasers at a minimum: at least the same number of lasers as power levels desired.

The present invention is advantageously practiced with diode lasers, where a number of individual emitters' output powers are combined to each port. A computer or control unit enables pre-setting of the desired output power for each port and calibration of each delivery system individually. It may also be advantageous to give 1 or 2 ports potentially higher output rating in order to enable the central fibers placed into a tumor to have higher power ranges than the more peripheral fibers.

A PDT treatment unit according to the present invention comprises a preselected number of diode units, each of which is optically connected to an output port located on the unit. As used in the present invention, a “diode unit” refers to a configuration consisting of one or more diode emitters. The diode unit may also contain additional features, including a substrate for securing one or more diode emitters, means to receive electric current, or means to regulate temperature such as a heat sink.

Each diode unit is connected to a power source to stimulate radiation emission. A control unit, preferably located on or in the treatment unit, allows a user to individually control the power supplied to each diode unit, to allow each diode unit to be individually adjusted to emit a desired power. The treatment unit is utilized by connecting a radiation delivery means to each output port. Each radiation delivery means, such as an optical fiber or optical fiber bundle, is then inserted into the tissue at a desired position to provide an optimal radiation pattern within the tissue.

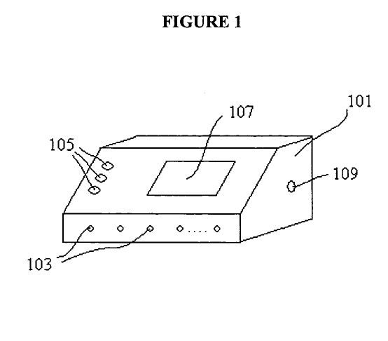

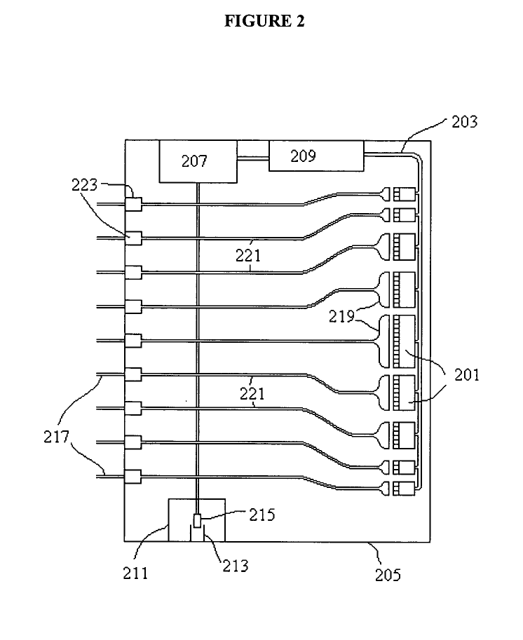

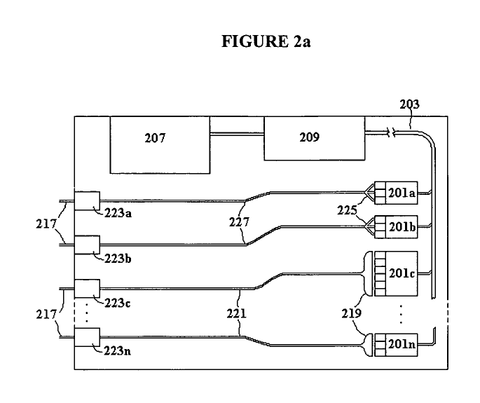

In a preferred embodiment, a PDT treatment laser unit is provided, as is shown in FIG. 1. Housing 101 contains numerous diode units (shown in FIG. 2), each of which is optically connected to one of a number of output ports 103. A control means or control unit (not shown), such as a computer, is connected to the power source and acts to control the amount of power to each diode unit. The control means includes input means 105 through which a user can enter desired power levels for each diode unit. In this example, input means is a series of buttons or a keypad that allows a user to enter desired power levels for each diode unit. In another embodiment, display screen 107 provides the user with information regarding the inputted and actual emitted power levels. Calibration port 109 is also provided to measure the radiation output from each delivery means and provide such information to the control unit, which may automatically calibrate the output for each diode unit based on desired output levels provided by the user, or which may allow the user to manually perform calibration.

In another embodiment, the control unit also comprises memory to store selected power levels for each diode unit. The control unit could also store information about the treatment, such as the type of treatment, tissue type, photosensitizer used (in the case of PDT), tumor size, and treatment duration, among others. These parameters may be stored so that, for future treatments, users can enter treatment parameters and the control unit can automatically set and maintain the required power settings, or display suggested power settings based on prior treatments, for a given treatment.

FIG. 2 illustrates the interior of a preferred treatment unit according to the present invention. Diode units 201, consisting of a preselected number of diode emitters, are connected to a power source (not shown) by electric leads 203. The power source may be outside of, within, or attached to housing 205. Control unit 207 is connected to power unit 209, to individually regulate the emitted power from each diode unit based on information received from the input means (shown in FIG. 1) or from optional calibration unit 211. Calibration unit includes calibration port 213 through the distal end of delivery means 217 is inserted, and measurement means 215, such as a photodiode, to measure radiation emitted from delivery means 217, and is connected to control unit 207 in order to relay calibration information to control unit 207. Calibration may be performed manually or automatically by control unit 207. Radiation from each diode unit 201 is coupled via beam combiners 219 into coupling fibers 221. Each coupling device 221 is connected to output port 223, to which delivery means 217, such as an optical fiber or fiber bundle, is optically connected so that radiation from diode units 201 is individually coupled into delivery means 217 for application to tissue.

FIG. 2a provides an alternative close-up view of an exemplary embodiment similar to that shown in FIG. 2, and illustrates additional features of the present invention. As shown in FIG. 2a, diode units 201a, b . . . n are preferably coupled, in this example, to corresponding output ports 223a, b . . . n. This configuration is not necessary, as each diode unit 201 may be coupled to any output port 223. For example, diode unit 201a may be connected to output port 223a, 223b, or any other desired output port 223. Also, not all diode units 201 need be coupled to an output port 223. It may be desirable to disconnect some diode units in the case where not all output ports 223 are to be used. Furthermore, FIG. 2a demonstrates that power unit 209 may be individually connected to each diode unit 201 to allow the power supplied to each diode unit 201 to be individually controlled via control unit 207. FIG. 2a also demonstrates alternative means to couple radiation from diode units 201 to output ports 223. For example, diode emitters in diode units 201 may be individually coupled to individual optical fibers 225 and combined in a single waveguide such as fiber optic bundles 227.

The examples described above are not limiting. Any number of diode units having different maximum power outputs may be utilized. They may be arranged in any pattern or configuration, depending on the embodiment. The number of output ports is also variable, and the number of diodes in any given diode unit may be varied. In another embodiment, interchangeable diode laser cartridges or laser modules may be used instead of simple arrays. Such diode laser cartridges are discussed in U.S. Pat. No. 6,421,361, and laser modules are discussed in U.S. Pat. No. 5,771,325, both of which have common inventorship with the present invention and are incorporated by reference herein.

As is shown in the example illustrated by FIG. 2, some of the diode units consist of larger diode emitter arrays than others. This provides a great deal of flexibility in choosing a radiation pattern. As is implied in FIG. 2, the above example may be ideally suited to setting a radiation pattern that consists of higher power radiation application toward the center of a tumor and lower power application away from the center and toward the periphery. Of course, this setup could be easily used to provide a variety of radiation patterns. For example, if it were desired that each source provide equal radiation power, simply lowering the power to the units having larger arrays to the power level of the smaller arrays would accomplish this configuration. Also, fewer than all of the sources in the unit need be activated, so that fewer numbers of radiation sources need be used if desired, such as for smaller tumors.

Diode units contemplated by the present invention may contain diodes of different types including diode lasers, light-emitting diodes, superluminescent diodes, Master Oscillator Power Amplifier (MOPA) diodes, and other types of high power laser diodes. The diode units containing the above and other types of diodes may contain one or more diodes, a diode bar or an array of diodes.

Beam combiners (exemplified as 219 in FIG. 2) may be any known means for coupling radiation from diodes or diode arrays into optical fibers or other waveguides. Exemplary beam combiners include microlens arrays, planar wave-guide combiners such as those described in U.S. Pat. No. 5,668,903, and rectangular waveguide combiners such as those described in U.S. Pat. No. 6,005,717, both of which have common inventorship with the present invention and are incorporated by reference herein.

Coupling devices (exemplified as 221 in FIG. 2) include optical fibers or optical fiber bundles, rectangular core optical fibers or bundles, rectangular waveguides, or other known waveguides capable of guiding treatment radiation.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A medical radiation device comprising:
multiple radiation output ports;
multiple radiation sources incorporated in said device and optically connected to said multiple radiation output ports; and
means to control power output levels emitted through each radiation output port, wherein said power output level can be controlled/set independently for each individual output port.

2. The medical radiation device according to claim 1, wherein said radiation source comprises a diode unit, wherein said diode unit comprises at least two diode emitters.

3. The medical radiation device according to claim 2, wherein said diode emitters are selected from the group consisting of a diode laser, a light-emitting diode, a superluminescent diode, a Master Oscillator Power Amplifier (MOPA) diode, and a tapered diode.

4. The medical radiation device according to claim 1, further comprising at least one power source connected to one or more said radiation sources and connected to said control means, wherein a power input from said at least one power source is controllable by said control means.

5. The medical radiation device according to claim 1, wherein said control means is a computer.

6. The medical radiation device according to claim 1, further comprising means to input power levels for each output port.

7. The medical radiation device according to claim 1, wherein said control means comprises a memory unit to store information on treatments.

8. The medical radiation device according to claim 7, wherein said treatments are selected from the group consisting of interstitial tumor therapy and photodynamic therapy.

9. The medical radiation device according to claim 1, further comprising a calibration means connected to said control means.

10. The medical radiation device according to claim 9, wherein said calibration means comprises:
a port for accepting an emission end of a radiation delivery device;
means to measure a power output from said emission end; and
means to transmit power output information from said measurement means to said control means.

11. The medical radiation device according to claim 10, wherein said measurement means is a photodiode.

12. A medical radiation system comprising:
the medical radiation device of claim 1; and
multiple radiation delivery devices, wherein each radiation delivery device is optically connected to one of said radiation output ports.

13. The medical radiation system according to claim 12, wherein said delivery device comprises a waveguide selected from the group consisting on an optical fiber and an optical fiber bundle.

14. The medical radiation system according to claim 12, wherein said delivery device comprises means to insert an emission end of said delivery device into a treatment area.

15. The medical radiation system according to claim 12, further comprising a calibration means connected to said control means.

16. The medical radiation system according to claim 15, wherein said calibration means comprises:
a port for accepting an emission end of a radiation delivery device;
means to measure a power output from said emission end; and
means to transmit power output information from said measurement means to said control means.

17. The medical radiation system according to claim 16, wherein said measurement means is a photodiode.
